(19) United States
(12) Patent Application Publication
(10) Pub. No.: US 2010/0114038 A1
(43) Pub. Date: May 6, 2010

(54) MEDICINE DELIVERY APPARATUS

(76) Inventor: Bernard Sams, Bedfordshire (GB)

Correspondence Address: YOUNG & THOMPSON, 209 Madison Street, Suite 500, Alexandria, VA, 22314, US

(21) Appl. No.: 12/513,415

(22) PCT Filed: Oct. 26, 2007
(86) PCT No.: PCT/GB2007/050656

§ 371 Date: Jul. 10, 2009

(30) Foreign Application Priority Data
Nov. 3, 2006 (GB) 0621901.8

(51) Int. Cl. A61M 5/315

(52) U.S. Cl. 604/211

(57) ABSTRACT

There is provided medicine delivery apparatus including a casing, a drive shaft which, in use, engages a medicine containing cartridge, an automatic driver, a user actuable trigger for causing the automatic driver to move the drive shaft through the casing, and a user actuable brake for allowing a user to control a rate of delivery of medicine.

### TECHNICAL FIELD

The present invention relates to an injection method and apparatus, and more particularly to an injection method and apparatus which provides for automatic medicine delivery and for variable dose setting.

### BACKGROUND

U.S. Pat. No. 5,743,889 and U.S. Pat. No. 6,899,698 describe a generally pen-like syringe that incorporates a dose metering device provided by a dose knob which is rotatable relative to the pen body to a position related to the dose of medicine to be injected. Within the body, there is a spring, a drum comprising a set of teeth and a grooved helical track, a drive rod arranged to push against a medicine cartridge and a lock that is arranged to releasably hold the drive rod. The lock includes a set of teeth arranged to engage with the drum teeth. The pen also includes a trigger that has a projecting cam follower. As the knob is rotated, the cam follower engages with the helical track and the drum undergoes rotational movement relative to the casing of the pen. This movement causes loading of the spring. The helical track ensures that the drum also undergoes axial movement along a main axis of the pen. The teeth of the drum move over the non-rotating teeth of the lock and disengage the lock from the drive rod as the lock teeth rise and fall during rotation. The axial movement of the lock corresponds to the axial movement of the drum. As the teeth are almost fully engaged between the lock and the drum, the lock grips the drive rod under the force of the spring. When the trigger is depressed, the cam follower is lifted out of engagement with the helical track, and the unwinding of the compressed spring pushes the lock and therefore the drive rod forward, thereby ejecting a dose of medicine. The front faces of the lock meet an internal stop fixed relative to the body. The drum and lock teeth are fully disengaged and the trigger cam follower can no longer engage with the helical track. A marking on the drum in this position is displayed through a window on the main body, which shows that the syringe is ready for further use.

A problem with these types of pen syringe is that the user has no control over the rate of ejection. The dose is ejected at a rate determined by the unwinding force of the spring. For larger doses, users may prefer to be able to limit the speed of medicine delivery.

### SUMMARY

According to a first aspect of the present invention there is provided medicine delivery apparatus comprising:
a casing;
a drive shaft which, in use, engages a medicine containing cartridge;
an automatic driver;
a user actuable trigger for causing the automatic driver to move the drive shaft through the casing; and
a user actuable brake for allowing a user to control a rate of delivery of medicine.

By “automatic driver”, it is meant a driver that does not require a motive force directly from a user. An automatic driver may be moved by a spring, a pre-cocked spring, a pneumatic force, and so on, and is distinct from a driver in a syringe that is pushed directly by a user.

It is preferred that the brake is disposed on the trigger, such that, in use, a pressure on the trigger operates the brake.

Preferably, the apparatus further comprises:
a dose setting knob;
driver teeth disposed on the driver;
a drum mounted in the casing and being rotatably coupled to the dose setting knob;
drum teeth disposed on the drum arranged to engage with the locking member teeth;
a helical track around an outer surface of the drum, the helical track arranged to engage with a cam follower disposed on the trigger; and
wherein, in use a rotation of the drum causes the drum teeth to move over the driver teeth, such that when the teeth are engaged the driver locks the drive shaft, and when the teeth are disengaged, the driver releases the drive shaft.

The brake may be arranged to restrict movement of the drum, thereby restricting movement of the drive shaft.

In order to facilitate moulding and assembly, the apparatus may further comprise a chassis located within the casing, the drum being partially located in and coaxial with the chassis, the chassis further comprising a projection arranged to contact the cam follower. In this instance it is preferred that the chassis comprises a chassis ramp disposed on the chassis and a drum ramp disposed on the drum, wherein, in use, a rotation of the drum relative to the chassis causes the ramps to co-operate to move the drum axially away from the chassis to a position in which the cam follower can engage with the helical track.

The apparatus may comprise a locking disc fixed relative to the casing, the locking disc having an opening through which the drive shaft can pass, the locking disc further comprising inwardly pointing fingers arranged to grip the drive shaft, such that the locking disk permits movement of the drive shaft in one direction only.

In order to allow the trigger to return to its original position after use, the apparatus may comprise an elastic overmoulding over the trigger, such that, in use, operation of the trigger deforms the overmoulding, and subsequent release of the trigger allows the overmoulding to revert to its original shape. Alternatively, the apparatus may comprise a leaf spring disposed between the trigger and the casing, the leaf spring biasing the trigger towards a rest position. Where a leaf spring is used, the brake may also act as the leaf spring.

In order to reduce the necessity for “air shots” or storing the medicine cartridge under pressure, the apparatus may comprise an opening in the dose setting knob, through which a user may manipulate the drive member to ensure contact with the medicine containing member.

According to a second aspect of the present invention, there is provided medicine delivery apparatus comprising:
a casing;
a drive shaft which, in use, engages a medicine containing cartridge;
an automatic driver;
a user actuable trigger for causing the automatic driver to move the drive shaft through the casing;
a cam follower located on the trigger, the cam follower arranged to engage with a helical track associated with the driver; and
a projecting surface fixed relative to the casing and arranged to contact the cam follower.

According to a third aspect of the invention, there is provided medicine delivery apparatus comprising:
a casing;
a drum rotatable within the casing and having a helical track formed on a surface thereof;
a cam follower which is engageable with said track to allow the drum to be screwed through the casing;
a first ramp located on the drum;
a second ramp fixed relative to the casing;
wherein, during dose setting, a rotation of the drum relative to the casing causes the ramps to co-operate to move the drum longitudinally within the casing, thereby bringing the cam follower into engagement with the helical track.

According to a fourth aspect of the present invention, there is provided medicine delivery apparatus comprising:
a casing;
a drive member which, in use, engages a medicine containing cartridge;
an automatic driver;
a user actuable trigger for causing the automatic driver to move the drive shaft through the casing;
an opening in the medicine delivery apparatus, through which a user may manipulate the drive member to ensure contact with the medicine containing member.

It is preferred that the opening is in a dose setting knob.

### BRIEF DESCRIPTION OF THE DRAWINGS

For a better understanding of the present invention and in order to show how the same may be carried into effect reference will now be made by way of example to the accompanying drawings in which:

FIG. 1 shows schematically a pen-type injector;

FIG. 2 shows a longitudinal cross-section through the injector of FIG. 1;

FIG. 3 shows an exploded perspective view of the injector of FIG. 1;

FIG. 4 is a perspective view of a dose knob of the injector of FIG. 1;

FIG. 5 is a perspective view of a lock of the injector of FIG. 1;

FIG. 6 is a perspective view of a drive rod of the injector of FIG. 1;

FIG. 7A shows a longitudinal cross-section through a drum of the injector of FIG. 1;

FIG. 7B is a perspective view of the drum of the injector of FIG. 1;

FIG. 8A is a perspective view of a chassis of the injector of FIG. 1′

FIG. 8B is a rear elevation view of the chassis of FIG. 8

FIG. 9 is a perspective view of a collar of the injector of FIG. 1;

FIG. 10A is a side elevation view of a trigger and drum of the injector illustrating a first alternative braking mechanism;

FIG. 10B is a side elevation view of a trigger and drum of the injector illustrating a second alternative braking mechanism; and

FIG. 10C is a side elevation view of a trigger and drum of the injector illustrating the braking mechanism of the injector of FIGS. 1 to 9.

### DETAILED DESCRIPTION

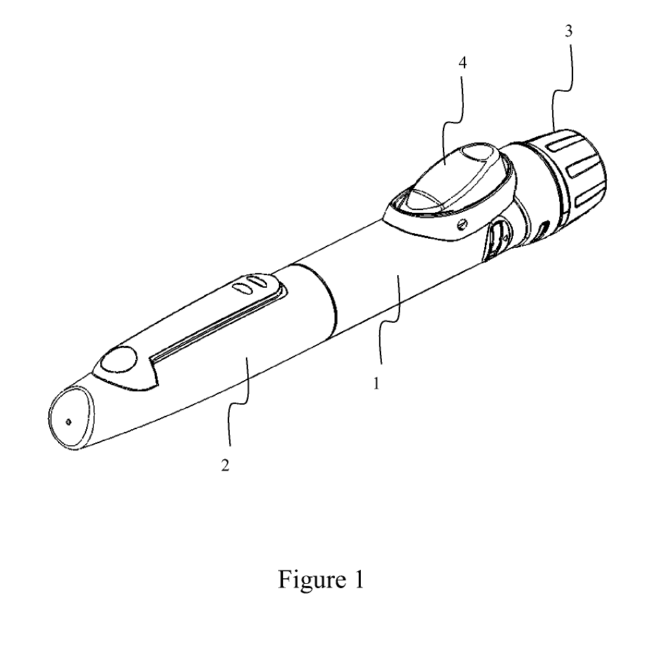

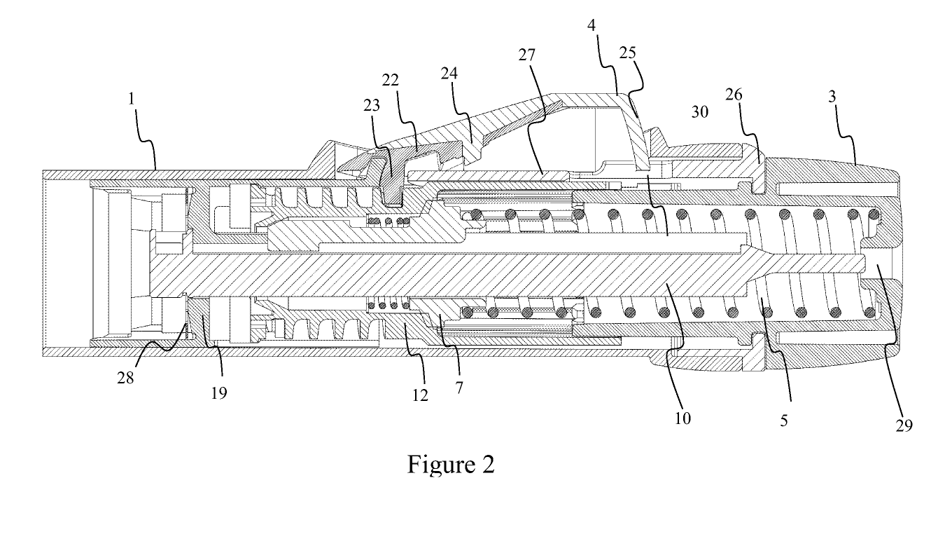

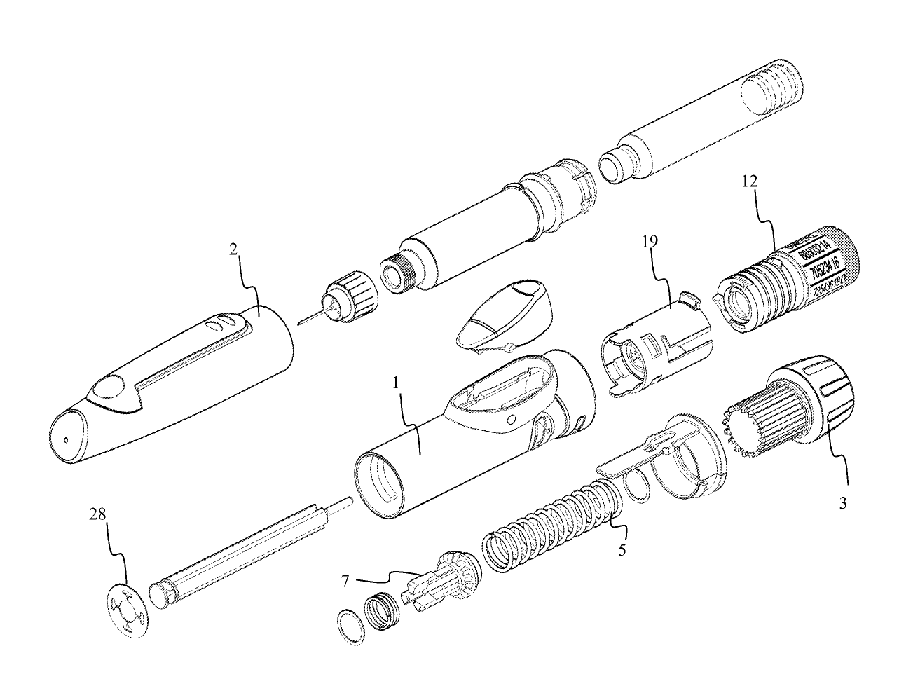

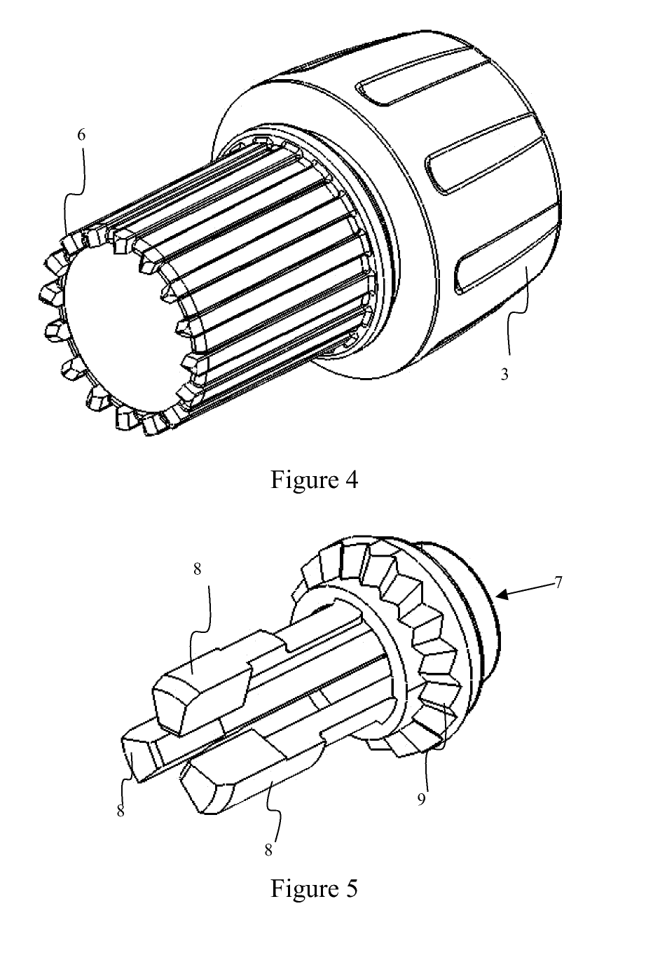

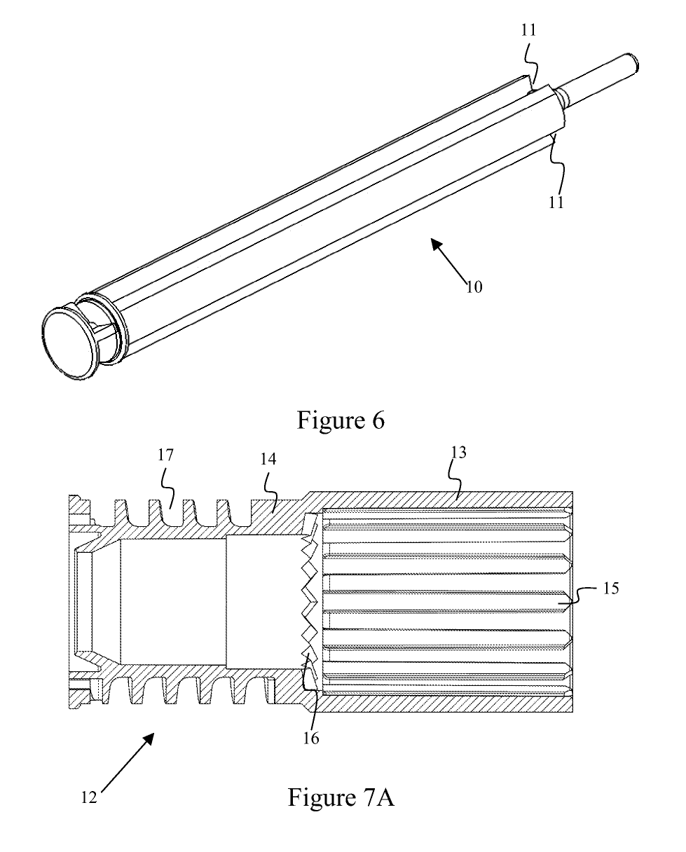

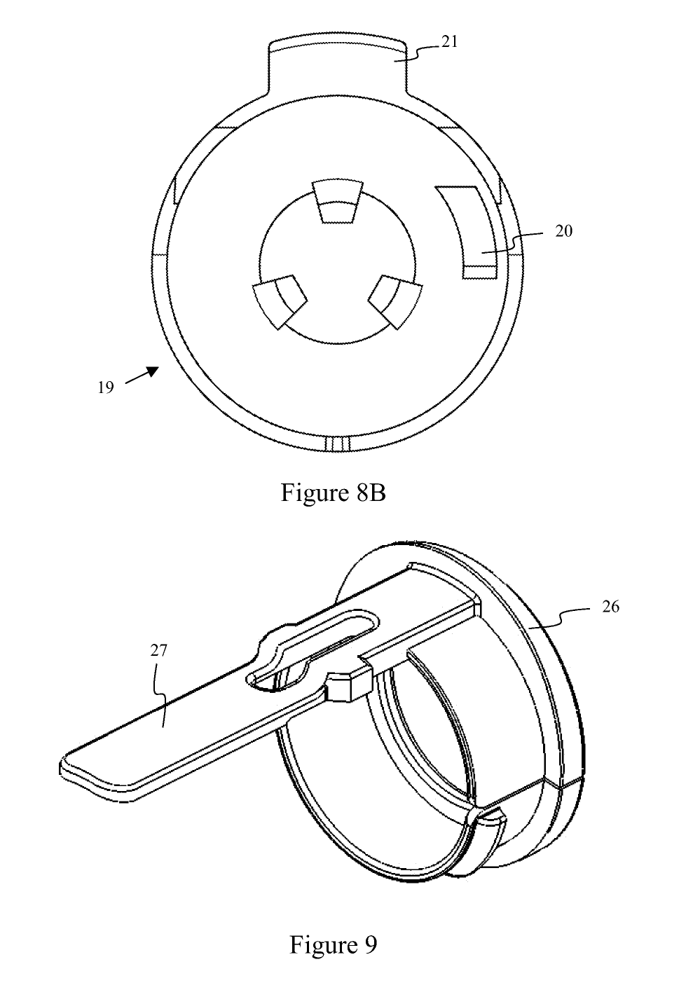

There is illustrated in FIGS. 1 to 9, and FIG. 10C, a pen-type injector having a user operable dose setting mechanism. It can be seen that the injector comprises a casing 1 and a cap 2, which snap fit together. A dose knob 3 is disposed at one end of the casing. A trigger 4 is also disposed on an outer surface of the casing. A cartridge housing (not shown) is secured to the end of the casing 1 opposite to the dose knob. The cartridge housing is arranged to receive a disposable medicine filled cartridge (not shown). Such a cartridge has a rubber bung sealing one end of the cartridge, with the other end being arranged to receive a disposable needle. The cartridge is typically multi-use, that is to say each cartridge contained multiple doses of medicine. A user attaches a new needle to the cartridge for each injection, and disposes of the cartridge or the whole device after all of the doses have been used.

The dose knob 3 comprises a gripping portion for a user to grip in order to rotate the dose knob. Extending from the gripping portion is a cylindrical portion having protruding splines 6. The gripping portion protrudes from the casing 1, whilst the cylindrical portion is located coaxially within the casing 1.

A compression spring 5 is located coaxially within the casing 1 and is arranged to provide the drive force for ejecting medicine from a loaded cartridge. The spring 5 abuts the dose knob 3 at one end, and its other end abuts a lock 7 located in the casing 1.

The lock 7 is located within and coaxial with the casing 1, and cannot rotate relative thereto. The lock 7 comprises an annular body defining an opening. Three locking legs 8 extend from the annular body in the direction of the main axis of the casing. The annular body also comprises a set of lock teeth 9. In this embodiment, three locking legs are provided, although it will be appreciated that any number of locking legs could be used.

A drive rod 10 is located in the casing 1. The drive rod 10 passes through the opening in the annular body of the lock 7. The drive rod 10 further comprises three channels 11 running along its length. Each channel 11 receives a locking leg 8, such that when the legs are in an unstressed state, the rod 10 can move freely through the lock 7. The drive rod 10 further comprises flanges at either end to prevent the lock 7 from slipping off the drive rod 10. At the end opposite the dose knob 3, the drive rod 10 abuts the rubber bung of the cartridge. The flange at the end of the drive rod 10 opposite to the end that contacts the rubber bung has a sloped surface such that when the lock 7 is assembled over the drive rod, the cone shape pushes the legs 8 such that they flex outwards and pass over the flange. However, once the locking legs 8 have passed over the flange they are restricted from passing back over the flange.

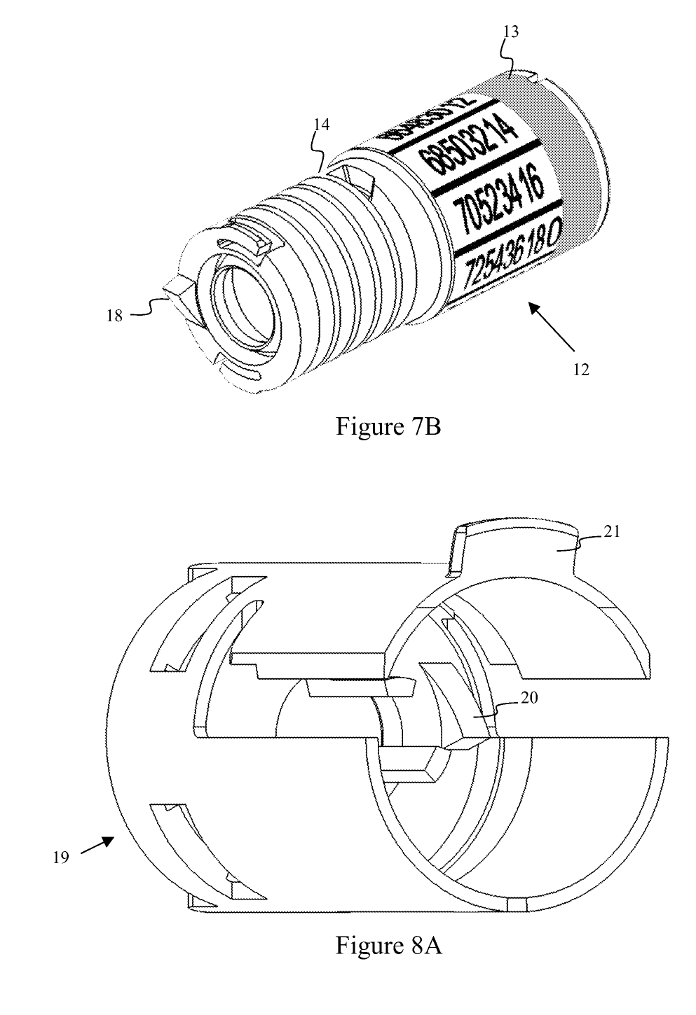

A drum 12 is also disposed coaxially within the main housing 1. The drum comprises a drum cylinder portion 13 and a drum track portion 14. The lock 7 is located coaxially within the drum 1, with the drive rod 10 passing through the lock as described above. The drum cylinder portion 13 comprises a multiplicity of drum splines 15 on an inner surface (see FIG. 7A). The drum splines engage with the dose knob splines 6, such that a rotation of the dose knob 3 causes a corresponding rotation of the drum 12, whilst allowing the drum 12 and the dose knob 3 to move axially relative to one another. A series of drum teeth 16 are also disposed on an inner surface of the drum 12, the drum teeth arranged to engage with the lock teeth 9. When the drum teeth 16 and the lock teeth 9 are engaged, the locking legs 8 push against angled inner surface of the drum 12, which stresses them and causes them to grip the drive rod 10. When the dose knob 3 is rotated, the drum teeth 16 move over the lock teeth 9, causing the lock 7 to move back and forward axially away from and towards the drum 12 and the locking legs 8 to release their friction grip on the drive rod 10. In order to bias the locking legs 8 in a gripping position, a strained biasing spring is attached at one end to the lock 7 and at the other end to the drum 12. This ensures that the lock 7 is urged towards the drum 12 and maintains a friction lock with the drive rod 10.

The drum cylinder portion 13 further comprises a series of numbers printed in a helical path along the outer surface of the cylinder. A window (not shown) in the casing 1 allows a user to see one of these numbers at a time. The numbers correspond to dose units of medicine that can be set by the user when turning the dose knob 3. In addition to the series of numbers, a green band is also printed on the drum cylinder portion 13. When the syringe is ready for use, and no dose has been set, the green band is visible through the window. When a dose is being set by rotating the dose knob 3, the green band is replaced by the numbers. Of course, any colour or pattern could be used in place of a green band to indicate that the syringe is ready for use.

The track portion 14 comprises a helical track 17 on its outer surface, and a projecting track ramp 18 at one end of the track portion 14. The track portion is coaxial with and is located in a chassis 19, formed as a separate component. The chassis 19 is a cylindrical component that is coaxial with and located within the casing 1 and cannot move relative thereto, and has an opening through which the drive rod 10 can move. The chassis 19 also comprises projections that engage with the drive rod channels 11, thereby ensuring that the drive rod 10 cannot rotate relative to the housing 1. The chassis 19 comprises a chassis ramp 20 arranged to co-operate with the track ramp 18, such that when the two ramps meet during rotational movement of the drum 12 relative to the casing 1 (the chassis 19 being fixed), the ramps slide across one another and cause an axial movement of the drum 12 away from the chassis 19 relative to the casing 1. The chassis further comprises a projecting reaction surface 21 arranged to engage with the trigger 4.

The trigger 4 allows the user to fire the dose of medicine. The trigger 4 co-operates with a lower trigger portion 22. The lower trigger portion has a projecting cam follower 23 that projects into the casing 1. The lower trigger portion 22 can move relative to the casing such that the cam follower 23 can move in and out of engagement with the helical track 17 of the drum 12. The trigger 4 comprises a trigger brake 24 that projects from an underside of the trigger 4 towards the casing 1.

The trigger 4 may be provided with a soft plastics overmoulded cover (not shown). An advantage of such an overmoulding is that when the trigger 4 is depressed, the overmoulding at the rear portion 25 of the trigger 4 is compressed. When the trigger 4 is released, the elasticity of the overmoulding urges the trigger 4 back towards its rest position. Alternatively, a leaf spring may be provided to return the trigger 4 to its rest position.

A collar 26 is provided attached to the end of the casing 1 from which the dose knob 3 protrudes. The collar 26 comprises an annular portion having an opening in which the dose knob 3 is located. The collar 26 further comprises a collar plate 27 which extends longitudinally within the casing 1 such that it is located between the trigger 4 and the drum cylinder 13.

When the pen syringe is assembled, the green band indicating zero dose can be seen through the window on the casing 1, indicating that the device is ready for use. When the user rotates the dose knob 3 to set the required dose, the dose knob splines 6 engaged with the drum splines 15 ensure that the drum 12 rotates. The cam follower 23 sits on an outer surface of the drum cylinder 13, and is not engaged with the helical track 17. As the drum 12 rotates within the chassis 19, the track ramp 18 moves around a base of the chassis. There is, however, no axial movement of the drum 12 relative to the chassis 19, and so no dose is being set. The track ramp 18 moves around the base of the chassis with no audible clicks, and the green band remains visible in the window of the casing 1. When the track ramp 18 comes into contact with the chassis ramp 20, co-operation between the ramps 18, 20 causes the drum 12 to move axially away from the chassis 19. The axial movement of the drum 12 away from the chassis 19 moves the helical track 17 such that the cam follower 23 of the lower trigger portion 22 drops into the helical track 17. The axial movement also moves the green printed band on the drum cylinder 13 away from the window in the casing 1, such that the first of the helically printed series of numbers is visible through the window. The use of the co-operating ramps 18, 20, allows the green band to be printed around the entire circumference of the cylinder drum so that when the pen syringe is ready for another dose to be set the green band is always visible through the window, no matter what rotational angle the drum 12 is disposed at relative to the casing 1.

Further rotation of the dose knob 3 causes rotation of the drum 12, and due to the engagement of the cam follower 23 with the helical track 17, this rotational movement is accompanied by further axial movement of the drum 12 away from the chassis 19, and consequent compression of the spring 5. This axial movement is accommodated within the casing by further telescoping of the drum 12 into the dose knob cylinder. The user can view the numbers printed on the drum cylinder 13 through the window in the casing, and set the dose required. Each increment in number is accompanied by an audible click caused by the drum teeth 16 moving over the lock teeth 9. If the user sets the dose too high, it can be reduced by counter-rotating the dose knob 3.

When the required dose has been set, the lock 7 locks the drive rod 10 in place relative to the drum 12. The user then inserts the needle into skin and presses the trigger 4 with a force sufficient to cause the lower trigger portion 22 to pivot and raise the cam follower 23 out of engagement with the helical track 17. The spring 5 unwinds and urges the lock 7 towards the chassis 19, thereby urging the locking legs into tight engagement with the drive rod 10. The drum 12, lock 7 and drive rod 10 are urged towards the chassis 19, thereby pushing the drive rod 10 against the rubber bung in the cartridge and causing ejection of the desired dose of medicine.

In order to control the rate of ejection of the medicine, the user exerts an extra force on the trigger 4, which pushes the trigger brake 24 against the collar plate 27. The collar plate 27 in turn presses against the drum cylinder 13. By varying the amount of force used to press the trigger 4, the user varies the amount of friction force between the drum 12, the collar plate 27 and the brake 24, and therefore controls the speed with which the drum 12 and consequently the drive rod 10 are urged forward by the unwinding of the spring 5. The collar plate 27 spreads the load of the force applied by the user over the drum, reducing wear on the components, and also reduces the risk that the numbers printed on the drum cylinder 13 are worn away.

The reaction surface 21 disposed on the chassis is in contact with the cam follower 23 of the lower trigger portion 22 to resist any undesirable force applied by the trigger 4 to the drum 12. The force of the spring 5 is borne by the reaction surface 21 rather than the pivots about which the trigger 4 moves, thereby reducing tolerance and wear problems.

If the pen syringe is mishandled, the drive rod 10 may slip out of contact with the rubber bung in the cartridge. If this occurs, then a user may believe that he is setting a higher dose than will actually be delivered, as the drive rod 10 must move to come into contact with the rubber bung before a dose is ejected. In order to prevent this, a resilient annular disc 28 having inwardly pointing fingers is located at the chassis 19. When the drive rod 10 is urged by the spring 5 to eject a dose of medicine, it passes through the centre of the resilient disc 28, splaying the fingers outwards. The splayed fingers grip the drive rod 10, thereby preventing any movement of the drive rod 10 back into the casing, that is to say the annular disc 28 forms a one-way “valve”. This also prevents re-use of the syringe. Of course, where re-use of the syringe is required then a releasable locking mechanism may be used in order to allow the drive rod 10 to move back into the casing 1.

When the pen syringe is assembled, it is assembled with a medicine cartridge in the cartridge housing. Due to the tolerances of parts, the end of the drive rod 10 may not engage the bung of the cartridge, and so the first dose ejected from the syringe will not be exactly the dose the user has set. The dose may either be too low, or the user may need to fire an “air shot” of a first dose of medicine to ensure that the drive rod is fully engaged with the rubber bung. Air shots are wasteful of expensive medicine, and if a user omits performing an air shot prior to the first dose, then the user may inject himself with an incorrect dose. One solution is to provide biasing means such as a spring to urge the drive rod onto the medicine cartridge. However, a pen syringe can be stored for periods of years before use, and it is not desirable to leave the cartridge under pressure for a long period of time. For this reason, the dose knob 3 is provided with an opening 29. Once the pen has been assembled, an elongate tool is pushed through the dose knob opening 29 against the drive rod 10, and the drive rod is pushed by the tool so as to be in contact with the cartridge. This ensures that the drive rod is at the base of the cartridge for the first dose to be ejected, but that the cartridge is not stored under pressure. Manufacturing tolerances are set to ensure that the drive rod 10 will not quite engage with the bung, rather than engaging with the bung to store the cartridge under pressure.

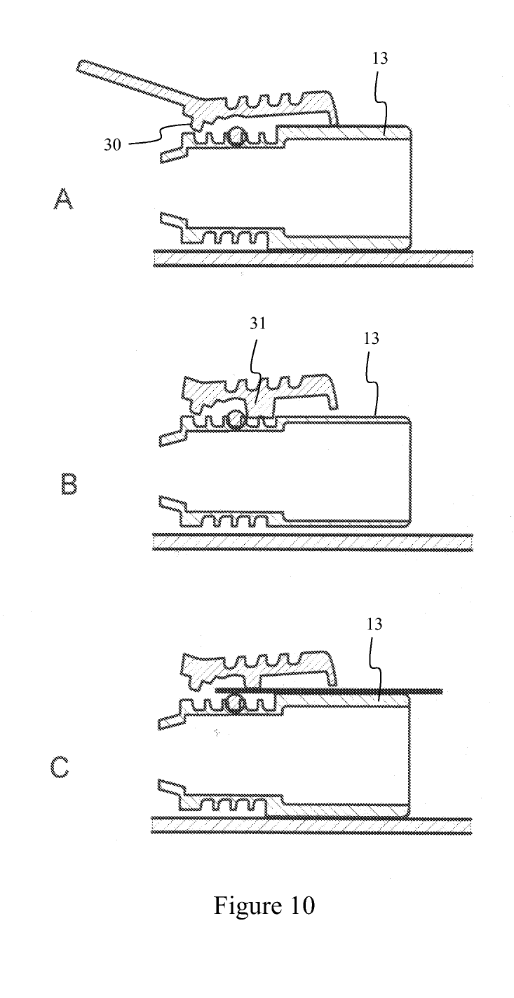

In a second specific embodiment, a collar plate 27 is not required. Referring to FIG. 10A, there is illustrated a trigger and a drum. The trigger is formed like a rocker switch having a peg 30 that is disposed over the helical track of the drum, by pressing the trigger such that the peg 30 comes into contact (but not engagement) with the helical track, the user can control the rate that the drum moves when ejecting a dose of medicine through friction between the peg 30 and the surface of the helical track.

According to a third specific embodiment, an alternative syringe is provided that also does not require a collar plate. Referring to FIG. 10B, a pad 31 is provided on the trigger which is larger than the width of the channel of the helical track 17. When a user depresses the trigger to fire a dose, a further user-applied force causes the pad 31 to come into contact with the outer surface of the drum track 14, and friction between the drum track 14 and the pad 31 reduces the speed at which the drum is urged forwards.

It will be appreciated by the person of skill in the art that various modifications may be made to the above described embodiments without departing from the scope of the present invention. For example, a leaf spring is referred to in the above description for returning the trigger to its rest position. A single part may be used to act as both the leaf spring and the brake. Furthermore, it will be appreciated that a chassis 19 is provided to assist in production and assembly, but as the chassis 19 is fixed relative to the casing 1, the functional parts of the chassis 19 such as the chassis ramp 20 and the reaction surface 21 could be formed integrally with the casing 1.

TABLE 1
List of parts

| Number | Part |
|---|---|
| 1 | Casing |
| 2 | Cap |
| 3 | Dose knob |
| 4 | Trigger |
| 5 | Spring |
| 6 | Splines |
| 7 | Lock |
| 8 | Locking legs |
| 9 | Lock teeth |
| 10 | Drive rod |
| 11 | Drive rod channels |
| 12 | Drum |
| 13 | Drum cylinder |
| 14 | Drum track |
| 15 | Drum splines |
| 16 | Drum teeth |
| 17 | Helical track |
| 18 | Track ramp |
| 19 | Chassis |
| 20 | Chassis ramp |
| 21 | Reaction surface |
| 22 | Lower trigger portion |
| 23 | Cam follower |
| 24 | Trigger brake |
| 25 | Trigger rear portion |
| 26 | Collar |
| 27 | Collar plate |
| 28 | Resilient disc |
| 29 | Dose knob opening |

1-15. (canceled)

16. Medicine delivery apparatus comprising:
a casing;
a drive shaft which, in use, engages a medicine containing cartridge;
an automatic driver;
a user operable trigger for causing the automatic driver to move the drive shaft through the casing;
a user operable brake for allowing a user to control a rate of delivery of medicine;
a dose setting knob;
a drum mounted in the casing and being rotatably coupled to the dose setting knob; and
a helical track around an outer surface of the drum, the helical track arranged to engage with a cam follower disposed on the trigger.

17. Apparatus according to claim 16, wherein the brake is disposed on the trigger, such that, in use, a pressure on the trigger operates the brake.

18. Apparatus as according to claim 16, comprising:
driver teeth disposed on the driver;
drum teeth disposed on the drum arranged to engage with the driver teeth;
wherein, in use a rotation of the drum causes the drum teeth to move over the driver teeth, such that when the teeth are engaged the driver locks the drive shaft, and when the teeth are disengaged, the driver releases the drive shaft.

19. Apparatus according to claim 18, wherein the brake is arranged to restrict movement of the drum, thereby restricting movement of the drive shaft.

20. Apparatus according to claim 18, comprising:
a chassis located within the casing, the drum being partially located in and coaxial with the chassis, the chassis further comprising a projection arranged to contact the cam follower.

21. Apparatus according to claim 18, comprising:
a chassis located within the casing, the drum being partially located in and coaxial with the chassis, the chassis further comprising a projection arranged to contact the cam follower;
a chassis ramp disposed on the chassis;
a drum ramp disposed on the drum;
wherein, in use, a rotation of the drum relative to the chassis causes the ramps to co-operate to move the drum axially away from the chassis to a position in which the cam follower can engage with the helical track.

22. Apparatus according to claim 16, further comprising a locking disc fixed relative to the casing, the locking disc having an opening through which the drive shaft can pass, the locking disc further comprising inwardly pointing fingers arranged to grip the drive shaft, such that the locking disk permits movement of the drive shaft in one direction only.

23. Apparatus according to claim 16, comprising an elastic overmoulding over the trigger, such that, in use, operation of the trigger deforms the overmoulding, and subsequent release of the trigger allows the overmoulding to revert to its original shape, thereby returning the trigger its original position.

24. Apparatus according to claim 16, comprising a leaf spring disposed between the trigger and the casing, the leaf spring biasing the trigger towards a rest position.

25. Apparatus according to claim 24 wherein the brake also acts as the leaf spring.

26. Apparatus according to claim 17, further comprising an opening in the dose setting knob, through which a user may manipulate the drive member to ensure contact with the medicine containing member.

27. Medicine delivery apparatus comprising:
a casing;
a drive shaft which, in use, engages a medicine containing cartridge;
an automatic driver;
a user actuable trigger for causing the automatic driver to move the drive shaft through the casing;
a cam follower located on the trigger, the cam follower arranged to engage with a helical track associated with the driver; and
a projecting surface fixed relative to the casing and arranged to contact the cam follower.

28. Medicine delivery apparatus comprising:
a casing;
a drum rotatable within the casing and having a helical track formed on a surface thereof;
a cam follower which is engageable with said track to allow the drum to be screwed through the casing;
a first ramp located on the drum;
a second ramp fixed relative to the casing;
wherein, during dose setting, a rotation of the drum relative to the casing causes the ramps to co-operate to move the drum longitudinally within the casing, thereby bringing the cam follower into engagement with the helical track.

29. Medicine delivery apparatus comprising:
a casing;
a drive member which, in use, engages a medicine containing cartridge;
an automatic driver;
a user actuable trigger for causing the automatic driver to move the drive shaft through the casing;
an opening in the medicine delivery apparatus, through which a user may manipulate the drive member to ensure contact with the medicine containing member.

30. Medicine delivery apparatus as claimed in claim 29, wherein the opening is in a dose setting knob.
